(19) United States
(12) Patent Application Publication
(10) Pub. No.: US 2026/0269021 A1
(43) Pub. Date: Sep. 10, 2026

(54) MODULAR PLATFORM FOR MEASURING VOLATILE ORGANIC COMPOUND PROFILES FROM EXHALED BREATH AND CORRELATING THOSE PROFILES TO PHYSIOLOGICAL AND PATHOLOGICAL STATES

(71) Applicants: Travis Miller, Poulsbo, WA (US); Rick Rainey, Las Vegas, NV (US); Sara Garland, Las Vegas, NV (US)

(72) Inventors: Travis Miller, Poulsbo, WA (US); Rick Rainey, Las Vegas, NV (US); Sara Garland, Las Vegas, NV (US)

(21) Appl. No.: 19/562,660

(22) Filed: Mar. 10, 2026

Related U.S. Application Data
(60) Provisional application No. 63/769,718, filed on Mar. 10, 2025.

(51) Int. Cl. G16C 20/20; G01N 1/22; G01N 1/40; G01N 33/497; G16H 50/20

(52) U.S. Cl. CPC G16C 20/20; G01N 1/22; G01N 33/4975; G16H 50/20; G01N 2001/2244; G01N 2001/227; G01N 2001/4088

(57) ABSTRACT

A modular breath analysis system is disclosed for analyzing volatile organic compounds in exhaled breath and correlating measured compound profiles with physiological or pathological states of a subject. The system may include a breath sampling subsystem, a breath conditioning subsystem, a sensor array, and a processing subsystem configured to generate a volatile organic compound profile and apply a correlation model. In some embodiments, a base instrument is configured to receive interchangeable sensing modules through mechanical, fluidic, and electrical interfaces. The sensing modules may include gas sensors, flow-routing structures, filters, membranes, identifiers, calibration data, or combinations thereof. Different sensing modules may be configured for different volatile organic compound panels associated with metabolic conditions, blood glucose estimation, respiratory conditions, infectious diseases, cancer, or other physiological or pathological states.

### COPYRIGHT NOTICE

Contained herein is material that is subject to copyright protection. The copyright owner has no objection to the facsimile reproduction by anyone of the patent document or the patent disclosure, as it appears in the United States Patent and Trademark Office patent file or records, but otherwise reserves all rights to the copyright whatsoever. The following notice applies to the software, screenshots and data as described below and, in the drawings attached hereto. All Rights Reserved.

### LEGAL NOTICE

All combinations and sub-combinations of the embodiments described herein are contemplated and considered part of the invention. Aspects and applications presented here are described below in the drawings and detailed description. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain, and ordinary meaning to the terms be applied to the interpretation of the specification and claims.

Further, the inventors are informed of the standards and application of the special provisions of 35 U.S.C. § 112(f). Thus, the use of the words “function,” “means”, or “step” in the Detailed Description or Description of the Drawings or claims is not intended to somehow indicate a desire to invoke the special provisions of 35 U.S.C. § 112(f) to define the systems, methods, processes, and/or apparatuses disclosed herein. To the contrary, if the provisions of 35 U.S.C. § 112(f) are sought to be invoked to define the embodiments, the claims will specifically and expressly state the exact phrases “means for” or “step for” and will also recite the word “function” (i.e., will state “means for performing the function of . . . ”), without also reciting in such phrases any structure, material, or act in support of the function. Thus, even when the claims recite a “means for performing the function of . . . ” or “step for performing the function of . . . ”, if the claims also recite any structure, material, or acts in support of that means or step, then it is the clear intention of the inventors not to invoke the provisions of 35 U.S.C. § 112(f). Moreover, even if the provisions of 35 U.S.C. § 112(f) are invoked to define the claimed embodiments, it is intended that the embodiments not be limited only to the specific structures, materials, or acts that are described in the preferred embodiments, but in addition, include any and all structures, materials, or acts that perform the claimed function as described in alternative embodiments or forms, or that are well known present or later-developed equivalent structures, materials, or acts for performing the claimed function.

### CROSS-REFERENCE TO RELATED APPLICATIONS

This application claims the benefit of U.S. Provisional Patent Application No. 63/769,718, filed Mar. 10, 2025, entitled “A SYSTEM AND METHOD FOR MEASURING VOLATILE ORGANIC COMPOUNDS IN EXHALED BREATH FOR DETERMINING BLOOD GLUCOSE LEVELS,” the entire contents of which are hereby incorporated by reference.

### TECHNICAL FIELD

This document relates generally to a medical device used to measure Volatile Organic Compounds (VOCs) to determine a patient's health. The present invention broadly relates to a medical device used to monitor an individual's health based off data collected from their breath. More specifically, data collected and analyzed using biomarkers based off volatile organic compounds (VOCs) expelled during the exhaled breath of a living organism. The present invention encompasses a method and an apparatus to measure and determine VOC concentrations to determine physiological or metabolic states including blood glucose level, as well as other indicators of various diseases and metabolic syndromes. In some embodiments, this device may detect volatile organic compound signatures associated with viral, bacterial, or fungal infections. The purpose of this device is to provide a non-invasive and convenient method for individuals to monitor or detect potential health risks or diseases.

### BACKGROUND

Presently, more than 38 million Americans have diabetes, which is about 11.6% of the U.S. population. This includes both diagnosed and undiagnosed cases, with the Centers for Disease Control and Prevention (CDC) estimating that 29.7 million cases (all ages) were diagnosed and another 8.7 million were undiagnosed in 2021. Another 97.6 million adults have prediabetes. Current estimates show that 1 in 3 Americans will develop diabetes sometime in their lifetime. Diabetes is a chronic (long lasting) health condition that affects how the body turns food into energy and results in too much sugar in the bloodstream. Over time, this can cause serious health problems and damage vital organs. Most people who have diabetes have a shorter life expectancy than people without the disease. Diabetes complications are increasing for young adults aged 18 to 44 and middle-aged adults aged 45 to 64. Nearly 1 in 5 adolescents aged 12 to 18 and 1 in 4 young adults aged 19 to 34 have prediabetes.

The total annual cost of diabetes in the United States is $413 billion USD, making it the most expensive chronic condition in our nation. Approximately $1 out of every $4 in the U.S. health care costs is spent on caring for people with diabetes. $307 billion is spent each year on direct medical costs and another $106 billion on reduced productivity. 48% to 64% of lifetime medical costs for a person with diabetes are for complications related to diabetes, such as heart disease and stroke.

In 2021 The International Diabetes Federation estimated that direct global health expenditure on diabetes for adults aged 20-79 was $966 billion. Projections show this could rise to $1.03 trillion by 2030. People diagnosed with diabetes have medical expenditures 2.6 times higher, on average, than those without diabetes. In 2022, the average annual medical expenditure was $19,736, with $12,022 attributed to diabetes.

CDC Strategies—CDC is working to help millions of Americans reduce their risk of type 2 diabetes and prevent or delay serious diabetes complications, which will save lives and lower medical costs. While some risk factors for type 2 diabetes, such as family history or age, cannot be changed, others can be avoided by maintaining a healthy weight and being physically active. To help prevent or delay type 2 diabetes, CDC's National Diabetes Prevention Program (National DPP) delivers an affordable, evidence-based lifestyle change program. Studies show that the lifestyle change program, which focuses on healthy eating and physical activity, can reduce the risk of type 2 diabetes by more than 50% for people at high risk. CDC and its partners are working to make the lifestyle change program available to more Americans. More than 750,000 adults had participated as of March 2024.

To prevent complications in people living with diabetes, CDC and its partners are working to expand access to and participation in diabetes self-management education and support (DSMES) services. DSMES services reach over 1 million people with diabetes each year. DSMES services help people with diabetes effectively manage their blood sugar, blood pressure, and cholesterol and get preventive care. Keeping diabetes under control through effective disease management can lower risks of diabetes complications. For example, effective blood sugar management can reduce the risk of eye disease, kidney disease, and nerve disease by 40%. Blood pressure management can reduce the risk of heart disease and stroke by 33% to 50%. Improved cholesterol levels can reduce cardiovascular complications by 20% to 50%. Detecting and treating early diabetic kidney disease by using kidney protective medicines that lower blood pressure can reduce decline in kidney function by 33% to 37%.

It is well understood in the medical industry that individuals with diabetes, whether type 1 or type 2, need to monitor their blood glucose levels regularly to ensure they stay within the target range. This routine testing, along with other daily practices, helps in making informed decisions about medication, diet, and lifestyle adjustments. Individuals with diabetes are typically required to check their blood glucose levels multiple times throughout the day. Blood glucose is typically monitored through a continuous glucose monitoring (CGM) system, or flash glucose monitoring systems. Fingerstick testing is most common with traditional meters, while CGM systems provide real time data through a sensor inserted under the skin.

Those with type 1 diabetes often require multiple daily injections of insulin or use an insulin pump. The dosage is based on blood glucose levels, carbohydrate intake, and anticipated physical activity. Some individuals with type 2 diabetes may also require insulin therapy, either alone or in combination with oral medications. Insulin may be administered through injections or specific insulin delivery devices known in the art.

Diabetics have dietary considerations consisting of managing carbohydrate and sugar intake. This is essential for blood sugar control. Individuals may use carbohydrate counting to match insulin doses with their meal plans. They also require regular visits to healthcare professionals to help monitor overall health and adjust treatment plans while addressing any concerns.

The present invention encompasses a system and methods for measuring volatile organic compounds (VOCs) produced in a human being's body. Endogenous volatile organic compounds are produced throughout the body and are picked up and distributed in the bloodstream. From the blood they exchange into air in the lungs and are then exhaled along with respiratory droplets and atmospheric gases. Exhaled VOCs contribute to biomarker discovery by providing a source of useful biomarkers with clear associations to the body's metabolism.

Breath Biopsy enables non-invasive collection and analysis of VOC biomarkers from breath. It takes roughly 1 minute for blood to flow around the entire circulatory system. By sampling breath for a minute or longer, even very low levels of VOC biomarkers from all parts of the body can be pre-concentrated, detected and identified. It's easy to increase sensitivity to aid in detecting subtle changes during the very early stages of disease simply by extending sample collection time.

It is known in the medical industry that diabetics produce VOCs which can either positively or negatively correlate to blood sugar levels, one of them being acetone. Acetone is a metabolic byproduct found in the exhaled breath and can be measured to monitor the metabolic degree of ketosis. In this state, the body uses free fatty acids as its main source of fuel because there is limited access to glucose. Monitoring ketosis is important for type I diabetes patients to prevent ketoacidosis, a potentially fatal condition, and individuals adjusting to a low-carbohydrate diet.

### Blood Alcohol Breathalyzers

A breathalyzer test measures blood alcohol content (BAC), which reflects the percentage of alcohol in a person's blood. Authorities can use BAC levels to gauge a person's level of intoxication. Following alcohol consumption, the body absorbs this chemical through the stomach lining into the bloodstream. As blood passes through the lungs, some alcohol evaporates and moves into the lungs.

The concentration of alcohol in the lungs relates to the concentration present in the blood. By using a partition ratio, it is possible to determine the BAC almost instantly from the air a person exhales rather than requiring a blood sample. The ratio of breath alcohol to blood alcohol is roughly 2,100:1. This means that roughly 2,100 milliliters (ml) of breath will contain the same amount of alcohol as 1 ml of blood.

Using the partition ratio, a breathalyzer can calculate a person's BAC. Generally, a breathalyzer is able to measure BAC due to a chemical reaction. The alcohol vapor in a person's breath reacts with an orange solution known as potassium dichromate. When alcohol is present, this solution turns green. This color change creates an electrical current, which the breathalyzer can convert into a value to determine the BAC.

Generally, there are two different types of breath analyzer tests: preliminary alcohol screening (PAS) tests and evidential breath tests (EBTs).

PAS tests refer to the small handheld devices that police may use in the field to determine a person's BAC. However, these machines are not always accurate. EBTs describe larger, stationary machines that are more reliable, which the police keep at the jail or station. A law enforcer may use a PAS machine prior to an arrest and then an EBT after the arrest to confirm the results.

Breathalyzers may also use different techniques to measure BAC. These can include electrochemical fuel cell breathalyzers, infrared optical sensor breathalyzers, semiconductor breathalyzers.

### Breath Analysis as an Additional Physiological Measurement Modality

Modem medical diagnostics rely on a variety of measurable physiological indicators, often referred to as biomarkers or biometric indicators. Examples of routinely measured physiological parameters include blood chemistry markers, heart rate, blood pressure, oxygen saturation, glucose concentration, and other clinical metrics obtained through blood tests or physiological monitoring devices.

These measurements provide valuable information regarding metabolic state, organ function, disease progression, and overall health status. However, many conventional diagnostic techniques rely on invasive sampling methods, including blood draws or other clinical procedures, which may limit the frequency with which measurements can be obtained.

Exhaled breath contains a complex mixture of volatile organic compounds (VOCs) produced through normal metabolic processes within the human body. Many of these compounds originate from biochemical pathways occurring in tissues, organs, and microbial populations within the body and are transported via the bloodstream to the lungs where they may be exhaled. As a result, the composition of exhaled breath may reflect metabolic activity occurring within the body.

Research has identified correlations between specific volatile compounds or patterns of volatile compounds in breath and various physiological and pathological states. These include metabolic conditions, inflammatory processes, respiratory disorders, infectious diseases, and other health-related conditions. Because breath sampling is non-invasive and can be performed repeatedly with minimal discomfort, breath analysis has been investigated as a potential diagnostic modality for monitoring physiological conditions.

Despite this potential, breath-based diagnostics have historically been limited by technical challenges including variability in breath sampling, environmental interference, humidity effects, and the need for sensing technologies capable of detecting low concentrations of volatile compounds.

Accordingly, there exists a need for systems capable of reliably measuring volatile compound profiles in exhaled breath and correlating those profiles with physiological conditions.

In certain embodiments described herein, a modular breath analysis platform is provided that enables measurement and analysis of volatile organic compound profiles in exhaled breath. By enabling detection and analysis of breath-derived volatile compounds, such systems may provide an additional class of physiological measurement that complements traditional biomarkers such as blood chemistry, heart rate, and oxygen saturation.

In this manner, analysis of breath VOC profiles may serve as a non-invasive measurement modality capable of providing insight into metabolic activity and physiological state.

### SUMMARY OF THE INVENTION

Disclosed herein are systems and methods for measuring volatile organic compound (VOC) profiles present in exhaled breath and correlating those profiles with physiological and pathological states of a subject, including metabolic conditions, blood glucose levels, disease states, and other health indicators.

Although the best understanding of the present invention will be had from a thorough reading of the specification and claims presented below, this summary is provided in order to acquaint the reader with some of the new and useful features of the present invention. Of course, this summary is not intended to be a complete litany of all of the features of the present invention, nor is it intended in any way to limit the breadth of the claims, which are presented at the end of the detailed description of this application.

The method and apparatus described e discloses a system and methods broadly relating to a medical device used to monitor an individual's health based on data collected from exhaled breath. More specifically, exhaled biomarkers comprising volatile organic compounds (VOCs) expelled during the exhaled breath of an individual. The present invention describes a method and device used to measure VOCs to determine glucose levels, and in some embodiments, indicators of various diseases comprising asthma, COPD, cystic fibrosis, obstructive sleep apnea syndrome, pulmonary arterial hypertension, breast, colon, gastric, head and neck, lung, and prostate cancer, neurodegenerative diseases such as Alzheimer's, multiple sclerosis, and Parkinson's diseases, hyperglycemia, infectious influenza A and B, parainfluenza 1, 2, 3, respiratory syncytial virus, human metapneumovirus, human rhinoviruses, tuberculosis, aspergillosis, among others, as well as gastrointestinal diseases such as bacterial overgrowth, fructose malabsorption, and Helicobacter pylori (H. pylori). The present invention can also be used to track metabolic or respiratory functions. In some embodiments of this invention, measurement of blood-borne volatile organic compounds occurring in the human exhaled breath is achieved through interchangeable sensor-heads and various forms of VOC preconcentration apparatus depending on the targeted ailment. Changes in volatile organic compounds are a result of metabolic changes or pathological disorders. The primary detection method of the present invention utilizes gas sensors comprising metal oxide sensors (MOS), photoionization detectors (PIDs), quartz crystal microbalance sensors (QCMS), surface acoustic wave sensors (SAWS), electrochemical sensors (ECS), nanomaterial based VOC/gas sensors (NMVSs), and tunable diode laser absorption spectroscopy (TDLAS, sometimes referred to as TDLS, TLS or TLAS), which in some embodiments are used in an array to detect and measure the concentration of a wider range of VOCs with better selectivity. In one embodiment the combination of sensors utilized is MOS and electrochemical sensors. MOS sensors detect a wide range of VOCs with high sensitivity, while electrochemical sensors could be used to specifically target gases like ethanol or acetone. In another embodiment PID and QCM sensors are utilized. The PID sensor is used for broad-spectrum detection of VOCs, while QCM sensors provide precise, real-time data on specific VOCs like acetone or isoprene. Various health conditions cause a rise in concentrations of certain VOCs. The best combination of sensors for detecting VOCs in human breath depend on the target VOCs, the required sensitivity, and the constraints of the device (e.g. size and power consumption). For general-purpose breath analysis, MOS sensors (e.g. for acetone and ethanol) and electrochemical sensors (e.g., for ammonia or ethanol) are commonly used due to their cost-effectiveness, reliability, and portability. However, some of the embodiments for detection of specific diseases require more specialized or highly sensitive sensor technology such as PID, QCM, or TDLAS. In some embodiments a dehumidifier in the device is used to dry breath samples.

In the embodiments utilizing any combination of an array of sensors, sensor fusion algorithms, machine learning, and artificial intelligence are employed.

When analyzing a gas sample for volatile organic compounds (VOCs) using various sensors, sensor fusion algorithms and filters are essential for integrating data from multiple sensors to provide a more accurate and robust result. Here are some commonly used sensor fusion algorithms that could be applied to VOC analysis:

### 1. Kalman Filter

Description: The Kalman filter is a powerful and widely used algorithm for sensor fusion when the system is subject to noise and uncertainties. It can be used to estimate the state of the VOC concentration from noisy sensor data over time.

Use case: If the VOC sensors have noise and the system dynamics can be modeled, Kalman filters can estimate the true concentration of VOCs by combining data from various sensors. The Kalman filter can work with both linear and nonlinear models (e.g., Extended Kalman Filter or Unscented Kalman Filter for nonlinear systems).

### 2. Particle Filter (Monte Carlo Localization)

Description: Particle filters are useful for nonlinear, non-Gaussian problems. It represents a probability distribution of the state (e.g., VOC concentration) using a set of particles that are propagated over time based on sensor measurements.

Use case: This method is ideal when the VOC sensors have highly non-linear behavior or if there are a variety of different sensor types with different uncertainties that need to be taken into account.

### 3. Principal Component Analysis (PCA)

Description: PCA is a dimensionality reduction technique that can be used to extract key features from sensor data when there are multiple sensors with high-dimensional data. PCA finds the most important underlying patterns (principal components) that explain the variance in the data.

Use case: If you have multiple sensors measuring different gases or environmental factors, PCA can be used to reduce the dimensionality of the data and highlight the main contributors to VOC measurements.

### 4. Artificial Neural Networks (ANN)

Description: ANN algorithms, such as multi-layer perceptron or deep learning models, can be used to model complex relationships between sensor readings and VOC concentrations. These networks can learn from large datasets and provide accurate predictions, especially in non-linear systems.

Use case: When dealing with highly nonlinear or complex relationships between the sensors and VOCs, ANNs can be trained on a large dataset to predict VOC concentration levels based on sensor inputs.

### 5. Support Vector Machines (SVM)

Description: SVMs can be used for classification and regression tasks. In the case of VOC analysis, SVMs could be used to classify different VOCs based on sensor data, or even to predict the concentration levels of VOCs.

Use case: SVMs are useful when you want to perform classification tasks (e.g., identifying the presence of certain VOCs) or regression tasks (e.g., estimating VOC concentration levels) using data from multiple sensors.

### 6. Multivariate Regression

Description: Multivariate regression algorithms can be used to create a model that relates multiple sensor readings to the concentration of VOCs in the sample. These methods can handle data from various sensors and provide continuous predictions for VOC levels.

Use case: If you have a known relationship between sensor outputs and VOC concentrations (perhaps from prior calibration), multivariate regression can be used to fuse sensor data into a single estimation of VOC levels.

### 7. Fuzzy Logic Systems

Description: Fuzzy logic systems can deal with uncertainty and imprecision in sensor data by using “fuzzy” membership functions. Instead of producing binary outputs (e.g., high/low concentration), fuzzy systems give a range of possible values that represent the uncertainty of the sensor data.

Use case: If sensors have high uncertainty, or if the VOC concentrations fall within a range that's difficult to categorize precisely, fuzzy logic systems allow for more flexible and interpretable predictions.

### 8. Bayesian Networks

Description: Bayesian networks represent probabilistic relationships between variables (e.g., VOC concentrations) and sensor outputs. They can be used to model and reason about the uncertainties and dependencies between different sensors and the VOC levels.

Use case: If you have prior knowledge about the interactions between sensors and VOCs, Bayesian networks can be used to update beliefs about VOC concentrations based on new sensor readings.

### 9. Weighted Average Fusion (WAF)

Description: This is a simple sensor fusion technique that involves assigning different weights to the sensor data based on their reliability or accuracy. The fused result is a weighted average of the sensor readings.

Use case: If certain sensors are known to be more reliable or accurate for certain VOCs or in specific environmental conditions, weighted average fusion can combine sensor data to give a more accurate estimate of VOC concentrations.

### 10. Data Association and Kalman Filtering for Multiple Sensors

Description: This algorithm focuses on sensor fusion in multi-sensor environments, where it's essential to identify and associate the readings of each sensor to specific components or VOCs in a complex mixture.

Use case: In applications where multiple sensors are used for detecting different components of a gas mixture, such as VOCs, the fusion algorithm can associate sensor outputs with the correct VOC species and combine their readings to yield a concentration estimate. In some embodiments multiple filters are implemented.

### Implementation Considerations:

Sensor Calibration: Before applying any fusion technique, ensure that the sensors are calibrated properly for the specific VOCs they will be measuring. Calibration can reduce the error from individual sensor outputs.

Sensor Characteristics: Some sensors might respond differently to various VOCs (e.g., metal oxide sensors vs. electrochemical sensors). The fusion algorithm should account for the sensor types and their specific characteristics.

In summary, choosing the best sensor fusion algorithm depends on the characteristics of your sensors (e.g., noise levels, sensor types), the nature of the VOCs you're detecting, and the complexity of the relationships between the sensor outputs and the VOC concentrations. For a high-performing system, you might combine multiple approaches (e.g., using PCA for dimensionality reduction followed by an ANN for prediction).

Machine learning algorithms and other forms of artificial intelligence (AI) may also be implemented.

Some other detection methods include at least one of optical sensors and AI machine learning to determine the presence of various VOCs, and biological organisms such as viruses, bacteria, and fungus.

Other features of the present invention will be apparent from the accompanying drawings and from the detailed description that follows. Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts.

### Disease Detection

The present invention relates to a novel system for detecting various diseases through the analysis of Volatile Organic Compounds (VOCs). More specifically, the invention encompasses a comprehensive method and apparatus for measuring and interpreting VOC patterns to identify specific diseases or health conditions.

Traditional methods of disease detection often rely on invasive procedures, time-consuming laboratory tests, and costly diagnostic equipment. There is a need for non-invasive, cost-effective, and rapid diagnostic tools that can detect diseases at an early stage. Recent research has shown a correlation between the presence of certain

VOCs in exhaled breath and specific diseases, indicating the potential for VOC analysis as a diagnostic method. The proposed invention comprises an integrated system for measuring and analyzing VOCs to detect various diseases and determine blood sugar. The system includes:

### Sample Collection Module:

Non-invasive sample collection from the patient's breath.
Integrated sensors for real-time monitoring of exhaled air.
Adjustable sampling parameters for optimal VOC capture.

### VOC Detection Unit:

Highly sensitive and selective VOC sensors.
Array of sensors to detect a broad spectrum of VOCs associated with different diseases.
Calibration mechanisms for accuracy and reliability.

### Data Processing and Analysis Module:

### User Interface:

Microprocessor-based data processing unit.
Machine learning algorithms for pattern recognition and disease classification.
Database of VOC profiles associated with various diseases for comparison.

### Advantages of the Invention

Non-Invasive: Eliminates the need for invasive procedures, reducing patient discomfort.
Early Detection: Enables early detection of diseases by capturing subtle changes in VOC patterns.
Cost-Effective: Provides a cost-effective alternative to traditional diagnostic methods.
Rapid Results: Delivers rapid and real-time results for immediate decision-making.

Applications: The invention has the potential for application in various fields, including but not limited to:
Respiratory diseases (e.g., asthma, chronic obstructive pulmonary disease).
Metabolic disorders (e.g., diabetes).
Infectious diseases (e.g., respiratory infections).

Conclusion: This patent disclosure outlines an integrated system that leverages VOC analysis for the early detection of various diseases. The invention's non-invasive nature, coupled with real-time analysis capabilities, makes it a promising diagnostic tool with broad applications in healthcare.

### Supporting and Relevant Info on Disease Detection by Measuring VOCs

Endogenous volatile organic compounds are produced throughout the body and are picked up and distributed in the bloodstream. From the blood they exchange into air in the lungs and are then exhaled along with respiratory droplets and atmospheric gases. Exhaled VOCs contribute to biomarker discovery by providing a source of useful biomarkers with clear associations to the body's metabolism.

Breath Biopsy enables non-invasive collection and analysis of VOC biomarkers from breath. It takes roughly 1 minute for blood to flow around the entire circulatory system. By sampling breath for a minute or longer, even very low levels of VOC biomarkers from all parts of the body can be pre-concentrated, detected and identified. It's easy to increase sensitivity to aid in detecting subtle changes during the very early stages of disease simply by extending sample collection time.

Lungs are very effective at exchanging chemicals with the bloodstream, including volatile metabolites and biomarkers that are generated even in the earliest stages of disease. It takes one minute for your blood volume to cycle around the average human body at once. By continuously preconcentrating exhaled chemicals we can sample and analyze the entire circulating blood volume.

Exogenous volatile organic compounds have a broader range of origins and have largely been overlooked as environmental contaminants in biological samples. More recently however, exogenous VOCs have become of increasing interest as many of them interact with biological systems and can also provide valuable information relevant to health and disease. For example, exogenous VOCs produced from the gut microbiome can be key health indicators.

Breath Sampling System: The device incorporates a breath sampling system that allows the user to exhale into the device's mouthpiece. The system ensures controlled airflow and prevents contamination to obtain a consistent and representative breath sample.

Gas Sensing Technology: The heart of the device lies in its advanced gas sensing technology, which is designed to detect and quantify select VOC levels in the breath. This technology utilizes sensitive and selective gas sensors capable of detecting trace amounts of VOC molecules.

Data Processing Unit: The device is equipped with a powerful data processing unit responsible for collecting, analyzing, and interpreting the data obtained from the gas sensors. The data processing unit employs complex algorithms to correlate VOC levels in the breath with physiologic states or diseases, taking into account individual variations and external factors.

Display and User Interface: The device features a user-friendly display screen that provides real-time measurements along with relevant information. The user interface allows for easy navigation and interaction with the device.

Operating Principle: In some embodiments, the VOC-based breathalyzer measurement device operates on the principle that acetone, a volatile organic compound, is produced in the human body as a metabolic byproduct when blood sugar levels are elevated. As glucose metabolism increases, acetone is released into the bloodstream and subsequently exhaled through the breath. By accurately measuring the concentration of acetone in the breath, the device can infer the corresponding blood sugar levels.

Limitations: While the VOC based breathalyzer offers a non-invasive and convenient approach to blood sugar monitoring, it is important to acknowledge its limitations:

Variability: Blood sugar levels can vary significantly throughout the day due to various factors such as diet, exercise, stress, and medication. The device's accuracy may be influenced by these variables.

Interference: Other factors, such as alcohol consumption and certain medical conditions, can influence acetone levels in the breath, potentially affecting the accuracy of the measurements.

Calibration and Accuracy: The accuracy of the device is crucial for its reliable performance. Calibration processes are undertaken during manufacturing and user setup to ensure accurate readings.

The device is designed to account for individual variations, environmental conditions, and other factors that may influence acetone levels in the breath. Regular calibration and periodic maintenance are recommended to maintain optimal performance.

### DETAILED DESCRIPTION

### 1. Definitions and Terminology

As used herein, the term “breath sample” refers to a gaseous sample obtained from exhalation of a human subject, including end-tidal breath, mixed expiratory breath, or time-averaged breath collected over a defined sampling interval.

The term “breath-emitted volatile organic compound” or “breath VOC” refers to a volatile organic compound that is present in exhaled human breath as a result of endogenous metabolic processes, blood-borne exchange in the lungs, microbiome activity, or disease-related biochemical pathways.

The term “sensor array” refers to two or more chemical, electrochemical, optical, or physical sensors configured to generate distinct response signals to one or more VOCs.

The term “interchangeable sensor head” or “sensor cartridge” refers to a removable and replaceable module containing one or more sensors, filters, membranes, or preconcentration elements configured to preferentially detect a selected subset of VOCs.

The term “selective membrane” refers to a physical or chemical barrier configured to preferentially permit diffusion of certain VOCs while reducing or preventing passage of interferent compounds, humidity, or particulate matter.

The term “correlation model” refers to one or more mathematical, statistical, or machine-learning algorithms configured to relate measured breath VOC signals to a physiological or pathological state, including blood glucose level, metabolic state, or disease classification.

### 2. System Overview

In one embodiment, the invention comprises a non-invasive breath analysis system configured to measure volatile organic compounds in exhaled breath and to determine a physiological state of a subject, including blood glucose level, metabolic condition, or presence of disease. Breath VOCs exhibit biological variability and overlapping concentration ranges across populations, therefore the present invention employs multi-VOC sensing, normalization, and temporal modeling, among other methods to generate correlation models using VOC profiles/“breath-prints”.

The system generally includes a breath sampling and conditioning subsystem, a VOC conditioning and selectivity subsystem, a sensor array subsystem, a data processing and correlation subsystem, a user interface and communication subsystem.

The system may be embodied as a handheld device, desktop device, wearable device, or clinical instrument, and may operate as a standalone unit or in communication with external computing systems.

### 3. Breath Sampling and Conditioning Subsystem

In one embodiment, the breath sampling subsystem includes a mouthpiece, nasal interface, or mask configured to receive exhaled breath from a subject. The subsystem may include one or more flow channels configured to control airflow rate and sample consistency.

### Example Operating Ranges:

Breath flow rate: 50-1000 mL/min
Sampling duration: 5 seconds to 5 minutes
Total sampled volume: 100 mL to 10 liters

In some embodiments, the system preferentially samples end-tidal breath, which more closely reflects alveolar gas exchange and blood-borne VOC concentrations. In other embodiments, mixed or averaged breath samples are used.

The breath conditioning subsystem may further include:
Temperature control (e.g., 20-45° C.)
Humidity management (relative humidity reduction from >95% to <30%)
Carbon dioxide measurement for normalization
Particulate and aerosol filtration

### 4. Selective VOC Conditioning and Membrane Filtering

In many applications, exhaled breath contains thousands of VOCs as well as water vapor and environmental contaminants. To improve selectivity and reduce interference, the invention may employ one or more selective membranes or filtering layers upstream of the sensor array.

Membrane embodiments may include:
Polymer membranes with selective permeability based on polarity or molecular size
Hydrophobic membranes for humidity reduction
Chemically functionalized membranes that preferentially absorb ketones or aldehydes
Multi-layer membrane stacks

### Example Membrane Properties:

Thickness: 1-500 microns
VOC permeability ratios (target:interferent): 2:1 to >100:1
Response time through membrane: <1 second to 30 seconds

In some embodiments, membranes are replaceable or interchangeable to tune selectivity for different disease states. For example, a membrane optimized for ketone VOCs may be used for diabetes monitoring, while a different membrane may be used for cancer-associated aldehydes or hydrocarbons.

### 5. Sensor Array Architecture

The sensor array subsystem comprises one or more sensors configured to detect breath VOCs. Sensors may be selected from, but are not limited to:
Metal oxide semiconductor (MOS) sensors
Electrochemical sensors
Photoionization detectors (PID)
Quartz crystal microbalance (QCM) sensors
Surface acoustic wave (SAW) sensors
Optical or infrared sensors (e.g., TDLAS)
Nanomaterial-based chemiresistive sensors

In many embodiments, cross-sensitivity is intentionally leveraged such that each sensor responds to multiple VOCs, producing a characteristic response pattern that is interpreted algorithmically.

### Example Sensor Operating Ranges:

Detection limits: 10 ppt to 10 ppm
Response times: 10 ms to 30 seconds
Operating temperature (MOS): 50-400° C.

### 6. Interchangeable Sensor Heads and Modular Architecture

In a preferred embodiment, the system employs interchangeable sensor heads configured to target different VOC panels associated with different diseases or metabolic states.

Each sensor head may include:
One or more sensors
One or more selective membranes or filters
Embedded identification data (e.g., EEPROM, RFID)
Calibration parameters

### Example Sensor Head Embodiments:

Metabolic head: optimized for ketones, aldehydes, alkanes
Cancer screening head: optimized for aldehydes, aromatics, hydrocarbons
Respiratory disease head: optimized for nitric oxide, ammonia, sulfur VOCs

Sensor heads may be disposable or reusable and may be keyed to prevent incorrect insertion.

An important note, removable sensing module, interchangeable sensing module, or interchangeable sensor heads are all terminology to describe a sensing module that can be readily be swapped with various base modules, later described in this specification.

In certain embodiments, the breath analysis device includes a modular sensor architecture comprising one or more interchangeable sensor heads configured to selectively detect one or more volatile organic compounds (VOCs) or families of VOCs associated with a physiological or pathological condition. The interchangeable sensor heads allow the device to be configured for different diagnostic applications without requiring replacement of the primary instrument.

The interchangeable sensor head may comprise a removable module containing one or more sensors, filters, membranes, flow channels, electronic circuitry, calibration data, or other components used for detection and analysis of volatile organic compounds. In various embodiments the sensor head may be implemented as a removable cartridge, replaceable module, microfluidic cartridge, slide-in tray, threaded module, snap-in module, or other mechanically removable component.

In some embodiments the sensor head mechanically couples to the breath analysis device using one or more of the following interfaces:
keyed mechanical connectors
bayonet connectors
threaded interfaces
snap-fit connectors
magnetic coupling mechanisms
sliding rail or tray interfaces
latch or lever locking mechanisms

The coupling mechanism may ensure proper alignment of airflow channels, sensor elements, and electrical contacts when the sensor head is installed.

In some embodiments the mechanical interface is configured to prevent insertion of incompatible sensor heads or to ensure correct orientation of the sensor head within the device.

In certain embodiments, the breath analysis device comprises a removable sensing module configured to couple to a base instrument and to provide one or more sensing, sample-conditioning, or sample-routing functions associated with analysis of exhaled breath. The removable sensing module, also referred to herein as an interchangeable sensing module, sensor head, sensing cartridge, diagnostic cartridge, sensing insert, sensor tray, or replaceable module. Unless otherwise indicated, these terms may be used interchangeably to describe a physically separable component configured to be selectively installed in, removed from, or exchanged with the base instrument.

The removable sensing module may contain one or more sensing elements, sample-conditioning structures, flow-routing structures, electronic circuits, identifiers, calibration data, or combinations thereof. In some embodiments, the removable sensing module is configured for single use or limited use. In other embodiments, the removable sensing module is reusable and is configured for repeated installation and removal.

In certain embodiments, the base instrument comprises common platform components including one or more of a user interface, power supply, processing subsystem, communication subsystem, display, housing, flow-control subsystem, and data-storage subsystem, particulate filtering, and interferent VOC filtering, while the removable sensing module provides application-specific sensing capability. In this manner, a common instrument may be selectively configured for detection of different volatile organic compound panels, disease states, physiological states, or monitoring applications through installation of different removable sensing modules.

### Removable Cartridges

In some embodiments, the removable sensing module is implemented as a removable cartridge. The removable cartridge may be a self-contained assembly inserted into a receiving portion of the base instrument and removed after use, replacement, calibration, or reconfiguration.

The removable cartridge may include one or more VOC sensors, one or more selective membranes, one or more sorbent or preconcentration materials, internal flow channels, one or more electrical contacts or communication interfaces, calibration or manufacturing metadata stored in memory, sealing features configured to align with one or more flow ports of the base instrument.

In certain embodiments, the removable cartridge is configured to provide a substantially enclosed sample pathway from an inlet interface to a sensing region. In some embodiments, this enclosed pathway reduces environmental contamination, moisture ingress, particulate contamination, or carryover between uses.

In some embodiments, the removable cartridge is disposable. In other embodiments, the removable cartridge is a durable replaceable component designed for repeated use over a defined service interval.

Cartridge-based diagnostic systems and removable analytical cartridges are widely used in adjacent diagnostic fields, including modular point-of-care and microfluidic systems, which supports using cartridge implementations as a broad architectural embodiment here.

### Snap-In Modules

In certain embodiments, the removable sensing module is implemented as a snap-in module. The snap-in module may be inserted into the base instrument and retained by a mechanical interference fit, latch, spring arm, detent, clip, or other snap-fit structure. Prior modular diagnostic systems explicitly describe interchangeably plugged-in or snap-fit cartridge/module arrangements, which supports this embodiment as a known and practical modular interface type.

A snap-in module may be advantageous where:
rapid exchange is desired;
the module is user-replaceable without tools;
a compact handheld device is preferred;
the module is intended to be inserted frequently.

In some embodiments, the snap-in module includes one or more keyed surfaces, chamfers, tabs, rails, slots, alignment pins, or asymmetrical housings configured to prevent incorrect orientation or installation of an incompatible module.

In some embodiments, installation of the snap-in module automatically establishes:
fluidic alignment with a breath sample flow path;
electrical connection with the base instrument;
loading of module-specific configuration data.

In certain embodiments, a snap-in module may be used for disease-specific VOC panels, allowing a user or clinician to exchange one module for another without replacing the primary instrument.

### Slide-In Trays

In some embodiments, the removable sensing module is implemented as a slide-in tray. The slide-in tray may be received by one or more guide rails, grooves, channels, or linear supports within the base instrument and may be inserted along a substantially linear path until reaching a seated position.
The slide-in tray architecture may be useful where: multiple sensors or subcomponents are arranged across a planar or elongated support;
electrical contacts are made at an end-of-travel position;
the module is relatively thin or panel-like;
serviceability and visual inspection are desirable.

In some embodiments, the slide-in tray comprises a tray body supporting multiple sensing elements distributed along one or more flow paths. In some embodiments, the tray also supports one or more sample-conditioning layers, membranes, sorbent regions, or reference sensors. In certain embodiments, the base instrument includes a closure, latch, or actuator that engages the tray after insertion to complete one or more fluidic, pneumatic, or electrical seals.

In some embodiments, the slide-in tray enables the base instrument to accept modules having different sensing layouts or different panel architectures while preserving a common instrument housing and processing platform.

The slide-in tray architecture may be useful where:
multiple sensors or subcomponents are arranged across a planar or elongated support;
electrical contacts are made at an end-of-travel position;
the module is relatively thin or panel-like;
serviceability and visual inspection are desirable.

### Microfluidic Cartridges

In some embodiments, the removable sensing module is implemented as a microfluidic cartridge.

Microfluidic cartridge architectures are well established in diagnostics because they permit controlled routing of small fluid or gas volumes through channels, chambers, valves, and sensing regions while integrating sample preparation and detection functions in a compact disposable or replaceable structure.

For the present invention, a microfluidic cartridge may be configured for gaseous breath analysis and may include one or more of:
microscale gas channels;
expansion chambers;
diffusion chambers;
flow restrictors;
passive valves or active microvalves;
micro-preconcentrators;
membrane regions;
mixing regions;
sample splitting junctions;
reference pathways;
sensor chambers.

In certain embodiments, the microfluidic cartridge is configured to receive a breath sample or a conditioned portion thereof and route the sample through one or more internal pathways before exposing the sample to one or more sensing elements. In some embodiments, one path routes sample to an active sensor array while another path routes sample to a reference or background sensor. In other embodiments, one or more internal chambers are configured to selectively retain, preconcentrate, dehumidify, or delay transport of selected compounds.

In certain embodiments, the microfluidic cartridge includes integrated sample-conditioning features tailored to a selected VOC panel. For example a metabolic-monitoring cartridge may include structures favoring transport or stabilization of ketones and related VOCs; a cancer-screening cartridge may include structures favoring aldehydes, hydrocarbons, or nonpolar compounds; a respiratory-disease cartridge may include structures favoring ammonia, sulfur compounds, or inflammatory breath markers.

Because microfluidic cartridge technology supports integration of sample processing with detection, this embodiment is particularly useful where the module itself performs part of the sample-conditioning function, rather than relying entirely on the base instrument.

### Mechanical Coupling Variants

In various embodiments, the removable sensing module may mechanically couple to the base instrument through one or more of:
keyed connectors
bayonet interfaces
threaded interfaces
snap-fit interfaces
sliding rail interfaces
magnetic coupling interfaces
latch interfaces
clamp interfaces

In some embodiments, the coupling mechanism establishes repeatable alignment of one or more of:
gas inlet and outlet ports
sensing regions
electrical contacts
optical paths
thermal interfaces

In certain embodiments, the coupling mechanism also creates a seal, including a gasket seal, compression seal, elastomeric seal, or interference seal, to reduce leakage or environmental contamination.

### Functional Partitioning Between Base Instrument and Module

In certain embodiments, the removable sensing module contains substantially all application-specific components, while the base instrument contains common reusable components. In other embodiments, sensing responsibility is partitioned between the base instrument and the module. For example the base instrument may contain general-purpose sensors and the module may contain disease-specific filters or membranes; the base instrument may contain heaters, pumps, or processors and the module may contain sensors and calibration data; the module may contain flow-routing and sample-preparation features while the base instrument contains the sensing electronics.

### Base Instrument Module

In some embodiments the device comprises a base instrument module configured to support one or more removable sensing modules.

The base instrument module may include:
processing subsystem (microcontroller, CPU, FPGA, SoC)
memory and data storage
power management subsystem
communication subsystem (Bluetooth, WiFi, USB, etc.)
user interface and display
airflow generation and flow control hardware
breath sampling interface
conditioning subsystems
calibration subsystems

The base module may further include environmental conditioning components configured to prepare breath samples prior to delivery to a sensing module. Such components may include:
humidity control systems
particulate filters
interferant removal membranes
pre-concentration elements
heating elements
temperature control elements
flow regulators and pumps

In certain embodiments the base instrument module performs primary breath sample conditioning before the sample reaches the removable sensing module.

Conditioning components may include:

In some embodiments the base module performs conditioning steps such as:
removal of water vapor
removal of particulate matter
removal of interferant gases
temperature stabilization
sample flow regulation
initial pre-concentration of VOCs

Conditioning components may include:
particulate filters
desiccant systems
Nafion humidity membranes
PTFE membranes
sorbent traps
micro-preconcentrators
heating elements for thermal desorption

This separation allows the removable sensing modules to focus primarily on detection rather than general sample preparation.

In some embodiments the removable sensing module interfaces with the base instrument through a combined mechanical, fluidic, and electrical interface.

The interface may include:
gas inlet ports
gas outlet ports
electrical contacts
communication bus connections
alignment features
sealing elements

The interface may be configured so that installation of the removable sensing module automatically establishes a fluidic pathway from the breath sampling subsystem to the sensing region, electrical connectivity to the processing subsystem, and loading of module-specific configuration parameters.

### Interchangeable Base Units

In certain embodiments the base instrument itself may also be interchangeable, in some embodiments a handheld consumer base unit may be utilized, in others, a clinical desktop base unit, a wearable base unit, or a research instrument base unit may be utilized. Each base unit may accept the same removable sensing modules but provide different computational compatibilities, power capacities, communication features, and airflow control systems.

### Distributed Processing Architecture

In some embodiments processing functions are distributed between the base module and the removable sensing module. For example, a base module may perform primary signal acquisition, preprocessing, device management, and user interface operations. A sensing module may include analog front-end circuitry, signal conditioning circuits, module-specific calibration data, and embedded microcontroller(s). In other embodiments, all processing occurs within the base module.

### Device Form Factors and Deployment Environments

In various embodiments, the breath analysis system described herein may be implemented in a variety of physical configurations depending on the intended application environment.

In certain embodiments, the system may be implemented as a benchtop or laboratory instrument configured for use in research laboratories, clinical facilities, or diagnostic testing environments. Such embodiments may include expanded sensing capabilities, larger sensor arrays, higher power consumption, or additional analytical instrumentation suitable for research and data collection purposes.

For example, a benchtop embodiment may include:
larger sensor arrays or multiple sensing modules
expanded conditioning subsystems
integrated pumps or flow control hardware
external calibration gas interfaces
connections to external computing systems for data analysis

In certain embodiments, the benchtop system may be used to collect large datasets of breath VOC profiles associated with physiological states, diseases, metabolic conditions, or environmental exposures.

In other embodiments, the breath analysis system may be implemented as a clinical diagnostic instrument suitable for deployment in healthcare environments such as hospitals, outpatient clinics, or diagnostic laboratories.

In still other embodiments, the system may be implemented as a portable or handheld device configured for point-of-care testing, home health monitoring, or field use.

Portable embodiments may include:
reduced-size sensing modules
battery-powered operation
wireless communication with external devices
simplified breath sampling interfaces

In some embodiments, the system may further be implemented as a wearable or personal monitoring device capable of periodically analyzing exhaled breath to monitor physiological conditions over time.

Because the system architecture described herein separates common platform components from interchangeable sensing modules, the same sensing modules may be used across multiple device form factors including research instruments, clinical analyzers, and portable devices.

### Population-Level Data Collection

In certain embodiments the system may be used to collect breath VOC datasets from populations of subjects in order to develop and refine correlation models between breath VOC profiles and physiological conditions.

Breath measurements may be collected from subjects in research studies, clinical trials, or longitudinal monitoring programs. The collected datasets may be used to train statistical models, machine learning models, or other analytical algorithms that correlate VOC patterns with physiological states.

### Electrical Interface Between Module and Base

In certain embodiments the removable sensing module communicates with the base instrument through one or more electrical interfaces including digital communication buses such as PC, SPI, UART, or other serial interfaces. In other embodiments the sensing module transmits analog sensor signals to an analog front-end located in the base instrument.

### Head Identification

In some embodiments the removable sensing module includes an embedded identifier configured to allow the base instrument to recognize the installed module. The identifier may include an EEPROM device, RFID tag, NFC element, resistor coding scheme, optical identifier, or other electronic identification mechanism.

### Breath Flow Control Hardware

The conditioning subsystem may further include active or passive flow control elements including micro-pumps, valves, flow restrictors, and controlled sampling channels.

### 7. Target Breath VOC Families

The invention is configured to detect breath-emitted VOCs, including but not limited to the following families:

### Ketones (Primary Metabolic Indicators)

Acetone
2-propanol (isopropanol)

### Aldehydes (Oxidative Stress and Lipid Peroxidation)

Acetaldehyde
Hexanal
Heptanal
Nonanal

### Alkanes and Isoprenoids

Pentane
Ethane
Isoprene

### Alcohols

Ethanol
Methanol
1-propanol

### Sulfur-Containing VOCs

Hydrogen sulfide
Dimethyl sulfide

### Nitrogen-Containing VOCs

Ammonia
Trimethylamine

### 8. Ketone VOC Physiology and Metabolic Correlation

Acetone is a volatile ketone produced primarily through hepatic ketogenesis during periods of altered glucose metabolism, insulin deficiency, or insulin resistance. Acetone enters the bloodstream and is exchanged into alveolar air in the lungs, where it is exhaled in breath.

### Example Breath Acetone Ranges:

Healthy individuals: 100-900 ppb
Metabolic stress/fasting: 500-2000 ppb
Diabetic ketosis: >1800 ppb to >10 ppm

The invention recognizes that breath acetone alone is insufficient to reliably determine blood glucose level across all subjects and conditions. Therefore, the system correlates acetone measurements with additional VOCs, ratios, and temporal trends.

### 9. Data Processing and Correlation Algorithms

The data processing subsystem receives signals from the sensor array and applies one or more correlation models to determine a physiological state.

Processing steps may include:
Baseline correction
Drift compensation
Humidity and temperature normalization
VOC ratio calculation
Temporal trend analysis

### Example Correlation Features:

Acetone: isoprene ratio
Ketone:aldehyde ratio
Post-prandial VOC decay rates
Overnight VOC accumulation trends

Algorithms may include multivariate regression, Kalman filtering, principal component analysis, neural networks, support vector machines, and Bayesian inference models. The output may include estimated blood glucose level (e.g., mg/dL or mmol/L), metabolic state classification, and confidence or reliability score.

### 9.1 Overview of the Data Pipeline

In one embodiment, the data processing subsystem receives raw sensor signals from the sensor array (and optionally auxiliary sensors such as humidity, temperature, pressure, and CO₂ sensors) and generates one or more outputs including: (i) an estimated blood glucose value; (ii) a metabolic state classification; (iii) a disease likelihood classification; and/or (iv) a confidence metric.

The processing subsystem may be implemented on an embedded microcontroller, a system-on-chip, a dedicated signal processor, a smartphone, or a remote/cloud computing system. In some embodiments, a portion of processing is performed locally for real-time feedback, while model refinement and longitudinal trend computation are performed remotely.
The processing chain may include one or more of the following stages:
Signal acquisition and digitization
Preprocessing and quality checks
Compensation/normalization for confounders
Feature extraction
Model inference
Post-processing and output reporting
Longitudinal personalization/model adaptation

This approach is consistent with known challenges in breath analysis involving humidity, temperature variation, VOC background changes, and sampling variability.

### 9.2 Signal Acquisition and Digitization

Raw sensor outputs may include resistance, conductance, current, voltage, frequency shift, phase shift, optical absorbance, or other transducer outputs depending on sensor type. Sampling rates may be selected based on response dynamics.

### Example, Non-Limiting Ranges:

Sampling frequency: 1 Hz to 10 kHz (e.g., 5-200 Hz for slow chemiresistors; higher for fast optical/IMS subsystems)
ADC resolution: 8-24 bits
Measurement window: 1 s to 300 s per breath event (or continuous monitoring)

### 9.3 Breath Event Segmentation and Quality Control

In some embodiments, the system detects a breath event and segments data into inhalation/exhalation phases. Breath event detection may be performed using flow sensors, pressure sensors, CO₂ sensors, or by detecting characteristic changes in humidity or sensor response slopes.

Breath quality checks may include, verifying a minimum exhalation duration (e.g., 2-20 seconds), verifying minimum flow volume (e.g., 50 mL to 3 L), confirming alveolar/end-tidal capture via CO₂ plateau detection, rejecting breath samples contaminated by ambient air leakage or incomplete exhalation.

End-tidal collection and standardized sampling methods are known to reduce variability between subjects and sessions.

### 9.4 Baseline Correction, Drift Correction, and Sensor Aging

Sensor arrays used in breath testing may exhibit baseline shifts and sensitivity changes due to aging, thermal cycling, exposure to complex mixtures, and surface chemistry changes. These effects create drift and calibration requirements.

Accordingly, the processing subsystem may apply one or more drift compensation methods including:
baseline subtraction using pre-breath ambient baseline windows (e.g., 1-60 seconds),
reference-channel subtraction (e.g., a “blank” sensor exposed to conditioned air),
periodic exposure to an internal reference gas or calibration source,
model-based drift adaptation, including domain adaptation and drift compensation algorithms used in electronic-nose systems.

### 9.5 Humidity, Temperature, and Ambient VOC Normalization

Breath is highly humid and contains variable temperature and background VOCs. In some embodiments, the system measures humidity and temperature and includes these parameters as explicit model inputs or uses them to normalize signals.

Normalization may include:
scaling sensor responses by measured relative humidity (RH) and temperature,
applying humidity compensation curves (sensor-specific),
using a humidity-reducing membrane or dryer and measuring residual RH to validate conditioning,
subtracting or ratioing signals relative to an “ambient” sample obtained immediately prior to exhalation.

### Example Environmental Ranges:

Ambient temperature: 0-40° C.
Breath channel temperature control: 20-45° C.
Residual RH after conditioning: 5-60% RH

Breath sampling studies emphasize that flow rate, humidity, and collection methodology significantly affect VOC recovery and measurement stability.

### 9.6 Feature Extraction from VOC Signatures

In one embodiment, the system extracts features from the sensor array responses to form a “breath print.”

Features may include:
peak amplitude, area-under-curve, rise time, decay time,
steady-state values at fixed times (e.g., 3 s, 10 s, 20 s),
derivatives and slopes,
ratios between sensors or between inferred VOC families,
response kinetics during different phases of exhalation (e.g., early vs end-tidal).

### Example Feature Vector Dimensionality:

5-500 features per breath event, depending on sensor count and derived kinetics. To reduce overfitting risk, the system may constrain feature count and sensor count and expand training datasets, consistent with known e-nose deployment considerations.

### 9.7 Correlation Models for Blood Glucose and Metabolic State

The invention recognizes (and your incorporated literature explicitly notes) that large datasets may show limited direct correlation between breath acetone and instantaneous blood glucose in certain populations (e.g., many type 2 cohorts), even when acetone ranges differ on average.

Therefore, in some embodiments, the system employs multi-variable and/or time-dependent models that correlate breath VOC signatures to blood glucose level, glucose trends (rising/falling), insulin resistance state, and ketosis risk. Models may include multivariate linear or nonlinear regression, partial least squares (PLS), principal components regression (PCR), support vector regression (SVR), random forest or gradient boosting regression, neural networks, Bayesian models using priors and uncertainty estimation or other models known in the art.

Outputs may include a numeric estimate of blood glucose (mg/dL or mmol/L) and a confidence score. Confidence may be computed from model uncertainty, input quality metrics, and distance-to-training distribution.

### Example Output Ranges:

Blood glucose reporting: 20-600 mg/dL (or equivalent mmol/L)
Confidence score: 0-1, 0-100, or categorical (low/medium/high)

### 9.8 Temporal Modeling and Contextual Correlation

Because breath VOC profiles are dynamic and influenced by meals, exercise, stress, and circadian patterns, the system may incorporate temporal context and trend-based inference. Breath VOC dynamics and the limitation of single-point measurements are widely recognized.

In some embodiments, the device records timestamps, meal markers, activity markers, medication/insulin events, and sleep state. In another embodiment, the device infers context based on VOC kinetics and breath parameters.

Temporal modeling may include moving averages across 2-200 breath events, Kalman filtering to fuse breath estimates with prior estimates, detection of post-prandial response curves (e.g., a VOC signature change occurring 0-180 minutes after meals), personalized baselines updated over 1 day to 6 months.

### 9.9 Multi-Disease Classification and Platform Models

For disease screening (e.g., cancer), the device may apply classification models trained on breath print patterns rather than single VOC measurements. Two complementary approaches are commonly used: MS-based identification/quantification and sensor-array pattern recognition (electronic nose).

In some embodiments, the system produces a disease likelihood score (e.g., probability), a triage recommendation (e.g., “screen positive—refer for confirmatory testing”), and a confidence metric.

### 10. Disease-Specific Embodiments

### 10.1 Diabetes and Metabolic Syndrome Embodiments

In one embodiment (primary embodiment), the system is configured to estimate blood glucose and/or metabolic state in a subject using breath VOC measurements. The system may use a metabolic sensor head optimized for ketones and related VOC families and apply a metabolic correlation model.

### Example Target VOC Families and Rationale

The metabolic head may be configured for detection of one or more of Ketones (e.g., acetone, 2-propanol) representing ketogenesis and altered glucose utilization, Aldehydes (e.g., acetaldehyde, hexanal, heptanal, nonanal) associated with oxidative stress and lipid peroxidation, and Alkanes/isoprenoids (e.g., pentane, ethane, isoprene) associated with lipid peroxidation and metabolic pathways.

Breath acetone ranges and thresholds for healthy vs ketosis states are widely discussed in literature (often ~0.3-1.0 ppm in healthy individuals, and higher in ketosis).

Ranges in healthy vs T2D cohorts across meal conditions, including broad ranges in T2D and the observation that correlation to blood glucose may not be direct.

In one embodiment, the system estimates blood glucose level by correlating breath acetone, aldehydes, and alkanes with known metabolic patterns and individualized baselines.

### Cancer Detection

In another embodiment, the system detects disease-specific VOC patterns associated with cancer, including elevated aldehydes and hydrocarbons, using a cancer-specific sensor head and classification model.

### Example Operating Methods

### Method of Estimating Blood Glucose

Collect breath sample
Condition breath sample
Measure VOCs using sensor array
Apply correlation model
Output estimated blood glucose

### Method of Disease Screening

Select disease-specific sensor head
Collect breath sample
Analyze VOC pattern
Classify disease likelihood

### Calibration and Quality Control

Calibration may be performed:
During manufacturing
Periodically by the user
Automatically using internal references

### Example Calibration Ranges:

Calibration gas concentrations: 50 ppb to 5 ppm
Recalibration intervals: daily to annually

### Example Multi-Parameter Correlation Strategies

Examples include estimating glucose using multi-VOC regression (ketone+aldehyde+alkane signatures), classifying metabolic state into categories such as “normoglycemic,” “hyperglycemic risk,” “ketosis risk,” correlating post-prandial response in VOC patterns to glycemic excursions, using personalized baselines and within-subject changes rather than absolute population.

### Example Operating Modes

Spot check mode: single breath event with quality gating
Trend mode: multiple breath samples overtime (e.g., 2-20 samples/day)
Continuous assisted mode: periodic sampling every 5-120 minutes

### 10.2 Cancer and Multi-Disease Screening Embodiments

In another embodiment, the system is configured to detect a breath VOC signature associated with cancer (or other diseases) using a disease-specific sensor head and a classification model.

Breath-VOC cancer detection is frequently described as relying on breath “profiles” and sensor-array pattern recognition, with ongoing work emphasizing validation, durability, and improved data processing.

Large-scale studies often implicate mixtures including aldehydes and hydrocarbons rather than a single unique VOC.

### 10.2.1 Example Cancer Sensor Head Configuration

The cancer head may be optimized for:
aldehydes and hydrocarbons (via selective membranes or recognition layers),
nonpolar VOC discrimination (via steric gating or tailored receptor layers),
ultra-low concentration detection (ppb-ppt range) using high-sensitivity transducers in some embodiments.

### 10.2.2 Example Use Cases

early screening triage (non-diagnostic, prompts confirmatory testing),
monitoring recurrence risk or treatment response,
multi-disease screening where the classifier outputs multiple disease likelihoods.

### 10.3 Infectious and Respiratory Disease Embodiments (Optional Platform Breadth)

In additional embodiments, the invention is configured to detect patterns associated with infectious diseases or respiratory disorders using appropriate sensor heads and classifiers, including VOC families associated with inflammation, microbiome activity, or airway oxidative stress. (This is platform-enabling and can be supported by later dependent claims without narrowing the core invention.)

### 11. Example Operating Methods

### 11.1 Method of Estimating Blood Glucose (Non-Limiting Example)

In one embodiment, a method of estimating blood glucose includes:
Initiating a measurement session and recording time/context (optional: meal, medication, exercise).
Collecting an exhaled breath sample through a mouthpiece, mask, or nasal interface.
Conditioning the breath sample using one or more of: humidity reduction, temperature stabilization, particulate filtration, VOC-selective membrane filtering, and/or preconcentration.
Sensing VOCs using a sensor array to produce sensor responses.
Performing preprocessing, including baseline correction and quality gating.
Computing features including sensor-response amplitudes, kinetics, and ratios.
Applying a correlation model to estimate blood glucose level and confidence.
Outputting results via display and/or transmitting to an external device.

### Example Ranges (Non-Limiting):

conditioning time: 0.1-30 seconds
breath collection time: 2-120 seconds
number of sensors: 2-64
number of VOC families analyzed: 1-6

### 11.2 Method of Monitoring Metabolic Trends

In another embodiment, the system generates a metabolic trend by:
collecting multiple breath events across a day,
updating a personalized baseline,
producing an estimated glucose trend and a metabolic state score.

### Example Ranges (Non-Limiting):

samples per day: 1-50
baseline adaptation window: 1 day to 90 days
trend smoothing window: 2-200 events

### 11.3 Method of Disease Screening Using Interchangeable Sensor Heads

In one embodiment, a method of disease screening includes:
Selecting a sensor head associated with a disease panel (e.g., diabetes vs cancer).
Reading sensor head metadata (ID, calibration, usage history).
Conditioning and analyzing breath with head-specific filtering.
Applying a disease classifier model corresponding to the sensor head.
Outputting disease likelihood and confidence.

This modular approach supports both platform scaling and safe operation across multiple disease panels.

### 12. Calibration, Quality Control, and Updates

### 12.1 Factory Calibration and Characterization

In one embodiment, sensors are calibrated during manufacturing using known test gases or gas mixtures. Calibration may include generating response curves at multiple concentrations and at multiple humidity/temperature conditions.

### Example Calibration Ranges:

calibration gas concentrations: 10 ppb to 100 ppm (compound-dependent)
number of calibration points per VOC: 2-20
humidity points: 0-95% RH
temperature points: 0-60° C.

Calibration data may be stored in the device or in the sensor head (e.g., nonvolatile memory) and retrieved during operation.

### 12.2 Field Calibration and User Calibration

The device may support periodic recalibration. In some embodiments, calibration is performed by:
exposure to a known calibration cartridge,
exposure to a “zero air” source (VOC-scrubbed air) for baseline restoration,
algorithmic recalibration using repeated measurements of stable reference conditions.

Your disclosure already states calibration is crucial and may be performed during manufacturing and user setup, accounting for individual variations and environmental conditions.

### Example Field Calibration Intervals:

baseline check: every use
calibration verification: weekly to quarterly
sensor head replacement: monthly to annually

### 12.3 Self-Test and Fault Detection

In one embodiment, the device performs self-tests including sensor open/short detection, heater integrity checks (MOS embodiments), flow-path blockage detection, humidity sensor plausibility checks, out-of-range VOC detection indicating contamination or device damage. The system may block reporting a glucose estimate if QC thresholds are not met.

12.4 Model updates one embodiment, the correlation model is versioned and updated as clinical datasets grow. Updates may be delivered:
via device firmware updates,
via smartphone application updates,
via cloud model deployment.

To maintain auditability (particularly for regulated contexts), the device may store:
model version ID,
calibration version ID,
sensor head ID,
time/date, measurement conditions, and quality metrics.

### 13. Alternative Embodiments and Variations

### 13.1 Sampling Variations

Alternative sampling approaches may include:
mouthpiece-based direct sampling,
mask-based sampling,
nasal sampling for specific applications,
bag-based collection for offline analysis,
controlled end-tidal collection devices.

Breath sampling approaches (whole breath vs end-tidal) and collection systems are known to influence VOC results, supporting the inclusion of multiple embodiments.

### 13.2 Sample Preparation and Signal Amplification Variations

In some embodiments, sample preparation may include micro-preconcentrators (μ-preconcentrators), micro-GC or micro-column separation, humidity control stages, integrated temperature control and μ-preconcentrators, micro-GC, or related microfabricated preparation devices to increase relevant VOC concentration and reduce humidity before delivery to an NMVS array.

### Example Ranges (Non-Limiting):

preconcentration factor: 2× to 10,000×
preconcentrator adsorption time: 1 s to 10 min
desorption time: 0.1 s to 60 s

### 13.3 Sensor Technology Variations

The sensor array may include any combination of semi-selective cross-reactive sensors (pattern recognition), highly selective lock-and-key sensors, hybrid arrays combining both approaches. This “hybrid array” approach is consistent with the concept that uncertain VOC prints favor semi-selective arrays, while well-defined low-concentration targets favor high-specificity architectures.

### 13.4 Module Interchangeability

Sensor heads may vary by number of sensors (one or more), filter/membrane stack composition, preconcentrator inclusion, intended disease panel, disposable vs reusable construction, keyed physical connectors and software authentication. In some embodiments, the device prevents use of a head outside its validated intended panel by reading the head ID and loading only approved models.

### 13.5 Data Pathway and Integration Variations

The device may display results locally, transmit to a smartphone, transmit to a clinician portal, integrate with other monitoring systems such as continuous glucose monitors (CGMs) to provide improved trend estimation and cross-validation, integrate with electronic health records or remote monitoring platforms.

### 14. Additional Alternative Embodiments and Variations

The invention is not limited to the embodiments described herein. Variations may include:
Different sensor technologies
Different VOC panels
Cloud-based processing
Integration with CGMs or electronic health records

1. A system for analyzing volatile organic compounds in exhaled breath of a subject to determine a physiological or pathological state of the subject, comprising:
a base instrument comprising:
a breath sampling interface configured to receive an exhaled breath sample from the subject;
a breath conditioning subsystem configured to condition the breath sample prior to analysis, the conditioning subsystem comprising at least one of humidity reduction elements, particulate filters, interferant-removal membranes, temperature control elements, or volatile compound preconcentration elements; and
a processing subsystem comprising at least one processor and memory storing instructions executable to process sensor data;
a removable sensing module selectively coupled to the base instrument through a mechanical, fluidic, and electrical interface;
wherein the removable sensing module comprises at least one sensor array comprising a plurality of gas sensors configured to detect a plurality of volatile organic compounds within the conditioned breath sample; and
wherein the processing subsystem is configured to:
receive sensor signals generated by the sensor array;
generate a volatile organic compound profile of the breath sample; and
apply a correlation model to determine the physiological or pathological state of the subject.

2. A breath analysis system comprising:
a breath sampling subsystem configured to receive exhaled breath from a subject;
a breath conditioning subsystem configured to modify at least one characteristic of the breath sample prior to analysis, the conditioning subsystem comprising at least one of humidity control, temperature control, particulate filtration, or selective membrane filtering;
a sensor array comprising a plurality of gas sensors configured to detect concentrations of a plurality of volatile organic compounds within the breath sample and generate a VOC profile; and
a processing subsystem configured to receive the sensor response signals and generate a volatile organic compound profile of the breath sample and correlate the profile with at least one physiological or pathological state of the subject.

3. The system of claim 1, wherein the processing subsystem is configured to execute the correlation model to correlate the volatile organic compound profile to at least one physiological state of the subject, wherein the physiological state comprises at least one of a metabolic condition, blood glucose level, disease state, or disease risk classification.

4. The system of claim 1 wherein the conditioning subsystem comprises a selective membrane configured to preferentially transmit a target volatile organic compound relative to an interferent compound.

5. The system of claim 4 wherein the selective membrane exhibits a permeability ratio between the target compound and the interferent compound of at least 2:1.

6. The system of claim 1 wherein the sensor array comprises at least two sensors selected from the group consisting of: metal oxide semiconductor sensors, electrochemical sensors, photoionization detectors, quartz crystal microbalance sensors, and surface acoustic wave sensors.

7. The system of claim 1 wherein the plurality of volatile organic compounds comprises at least two compounds selected from the following families consisting of: ketones, aldehydes, alkanes, alcohols, sulfur-containing compounds, nitrogen-containing compounds.

8. The system of claim 1 wherein the physiological state comprises blood glucose level.

9. The system of claim 1 wherein the volatile organic compounds comprise acetone and at least one additional volatile organic compound selected from isoprene, pentane, or acetaldehyde.

10. The system of claim 1 wherein the processing subsystem determines the physiological state using temporal changes in volatile organic compound concentrations.

11. The system of claim 1 wherein the removable sensing module comprises an interchangeable sensor head configured for detection of a disease-specific volatile organic compound panel.

12. The system of claim 11 wherein the interchangeable sensor heads are configured to detect volatile organic compound panels associated with at least one of metabolic disorders, cancer, respiratory diseases, or infectious diseases.

13. The system of claim 1 wherein the removable sensing module comprises a microfluidic cartridge including one or more microchannels configured to route the breath sample to sensing regions.

14. The system of claim 1 wherein the removable sensing module includes an electronic identifier configured to identify the sensing module to the base instrument.

15. A method of determining a physiological state of a subject using volatile organic compounds in exhaled breath, comprising:
collecting an exhaled breath sample from the subject;
conditioning the breath sample to reduce at least one of humidity, particulate matter, or interfering volatile compounds;
detecting a plurality of volatile organic compounds in the conditioned breath sample using a sensor array;
generating sensor response signals corresponding to the volatile organic compounds;
extracting a volatile organic compound profile from the sensor response signals; and
applying a correlation model to the volatile organic compound profile to estimate the physiological state of the subject.
